(19) United States
(12) Patent Application Publication
(10) Pub. No.: US 2023/0365552 A1
(43) Pub. Date: Nov. 16, 2023

(54) LYSERGIC ACID DERIVATIVES AND METHODS

(71) Applicant: The Florida State University Research Foundation, Inc., Tallahassee, FL (US)

(72) Inventor: Joel Michael Smith, Tallahassee, FL (US)

(21) Appl. No.: 18/316,755

(22) Filed: May 12, 2023

Related U.S. Application Data
(60) Provisional application No. 63/341,162, filed on May 12, 2022.

(51) Int. Cl. C07D 457/02; B01J 23/44

(52) U.S. Cl. CPC C07D 457/02; B01J 23/44

(57) ABSTRACT

Methods of preparation of lysergic acid and derivatives thereof. Methods of using lysergic acid and derivatives thereof, such as methods of treating neurodegenerative disorders. Derivatives of lysergic acid and pharmaceutically acceptable salts thereof.

### CROSS-REFERENCE TO RELATED APPLICATIONS

This application claims priority to U.S. Provisional Patent Application No. 63/341,162, filed May 12, 2022, which is incorporated by reference herein.

### FIELD OF THE DISCLOSURE

The present disclosure is directed towards methods of preparation of lysergic acid and derivatives thereof. The present disclosure is further directed towards novel derivatives of lysergic acid. The present disclosure is also directed to methods of treatment.

### BACKGROUND OF THE DISCLOSURE

Since Hofmann's discovery of lysergic acid diethylamide (LSD) in 1938, the medicinal applications of this natural product derivative have been considered intriguing and controversial. For instance, Sandoz Laboratories heralded LSD as “a cure for everything” in the 1940s, while the U.S. congress, in partial response to counter-culture of the 1960s, made its possession and use illegal in 1968.

Despite this past, however, some ergoline derivatives, such as pergolide and lisuride, have found their way to the clinic for the treatment of Parkinson's disease and migraines. These ergoline structures, in addition to the psychedelics dimethyltryptamine (DMT) and 2,5,-dimethoxy-4-iodoamphetamine are ligands for the 5-HT_{2A} GCPR, a key receptor responsible for many downstream neuropharmacological phenotypes. Because of LSD's therapeutic potential, several X-ray crystallographic structures have recently been obtained that permit the design of 5-HT2A ligands capable of novel neuropharmacology.

There are many known methods for synthesizing lysergic acid, either asymmetrically or racemically. Many of the approaches, however, are not reproducible and/or are extensive in terms of the number of steps required. These disadvantages can add to extra waste generated by the methods' implementation, in addition to limiting the methods' viability and efficiency towards the production of particular analogs.

There remains a need for a practical synthesis of lysergic acid and derivatives thereof, including diverse LSD derivatives.

### SUMMARY

Described herein are compounds, including lysergic acid and lysergic acid derivatives, and methods of making compounds, such as lysergic acid and derivatives thereof. The synthetic brevity and synthetic malleability of embodiments of the methods described herein may allow for the development of non-natural analogs that can aid in the discovery of novel compounds with potentially important therapeutic indices for treating various neurodegenerative diseases, psychological, cognitive, behavioral, and/or mood disorders.

In one aspect, provided herein is a method to synthesize lysergic acid or derivatives thereof, such as from simple aromatic precursors. The lysergic acid derivatives may include those bearing substitution on the benzenoid ring of an indole nucleus of lysergic acid. In some embodiments, the method includes the coupling, dearomatization and cyclization of a halopyridine with a 4-haloindole derivative in six total synthetic steps from commercially-available starting materials. In some embodiments, the methods include providing a compound of formula (A)—

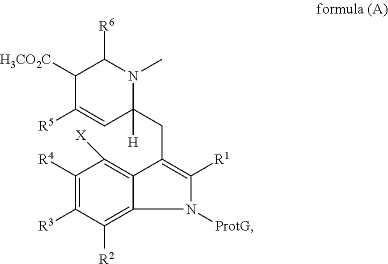

wherein ProtG is a protecting group, X is a halogen, and R¹-R⁶ are independently selected from hydrogen, hydroxy, methoxy, a halogen, or a C₁-C₂₀ hydrocarbyl, wherein, as defined herein, the C₁-C₂₀ hydrocarbyl is unsubstituted or substituted; and contacting the compound of formula (A) and an annulating agent to produce an annulated compound. The methods also may include contacting the annulated compound and an agent effective to remove the protecting group.

In another aspect, compounds or pharmaceutically acceptable salts or solvates thereof, and pharmaceutical formulations including the compounds or pharmaceutically acceptable salts or solvates thereof are provided. In some embodiments, the compounds or pharmaceutically acceptable salts or solvates thereof include those of formula (I) or formula (I′):

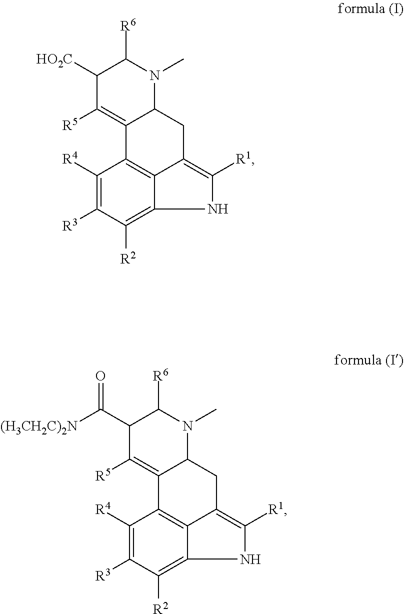

wherein R¹-R⁶ are independently selected from hydrogen, hydroxy, methoxy, a halogen, or a C₁-C₂₀ hydrocarbyl, wherein, as defined herein, the C₁-C₂₀ hydrocarbyl is unsubstituted or substituted. In some embodiments, at least one of R¹-R⁶ is not hydrogen.

In another aspect, provided herein are methods of treating neurodegenerative disease in a patient in need thereof. In some embodiments, the methods include administering to a patient suffering from neurodegenerative disease an effective amount of a compound or composition described herein or a pharmaceutically acceptable salt or solvate thereof. In some embodiments, the compounds and compositions described herein can be used to treat one or more neurodegenerative diseases and/or as intermediates and precursors for the production of lysergic acid derivatives, such as prodrugs, for treatment of one or more neurodegenerative diseases.

Other objects, features and advantages of the methods and compounds described herein will become apparent from the following detailed description and appended claims. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

In one embodiment provided herein is a method of synthesis of lysergic acid from simple aromatic precursors. In some embodiments, the method includes synthesizing derivatives of lysergic acid. In some embodiments, the compounds as described herein can be used in treating neurodegenerative diseases including, but not limited to, Alzheimer's disease, Parkinson's disease, Huntington's disease, Multiple Sclerosis, and Amyotrophic Lateral Sclerosis. In some embodiments, the compounds as described herein can be used in treating psychological, cognitive, behavioral, and/or mood disorders. In some embodiments, provided herein is a method of treating neurodegenerative disease in a patient in need thereof.

In some embodiments, the compounds of this disclosure or a pharmaceutically acceptable salt or solvate thereof, are administered in an amount of greater than about 30 μg, 40 μg, 50 μg, 60 μg, 70 μg, 80 μg, 90 μg, or 100 μg. In some embodiments, the compounds of this disclosure or a pharmaceutically acceptable salt or solvate thereof, are administered in an amount of 100 μg.

In some embodiments, the compounds of this disclosure or a pharmaceutically acceptable salt or solvate thereof, are administered in an amount of greater than about 0.01 mg, 0.05 mg, 0.1 mg, 0.5 mg, 1 mg or 10 mg.

In some embodiments, the compounds of this disclosure or a pharmaceutically acceptable salt or solvate thereof, are administered in an amount of greater than about 20 mg, 60 mg, 100 mg, 200 mg, 400 mg, or 500 mg.

In some embodiments, the compounds of this disclosure or a pharmaceutically acceptable salt or solvate thereof, are administered in an effective amount from of about 10 μg to about 500 μg mg per kg of the patient's body weight (mpk). In some embodiments, the compounds of this disclosure, or a pharmaceutically acceptable salt, or solvate thereof, can be administered in a regimen. The regimen can be structured to provide therapeutically effective amounts of the compounds over a predetermined period of time (e.g., an administration time). The regimen can be structured to limit or prevent side-effects or undesired complications of the compounds disclosed herein. Regimens useful for treating neurodegenerative diseases, psychological, cognitive, behavioral, and/or mood disorders can include any number of days of administration which can be repeated as necessary. Administration periods can be broken by a rest period that includes no administration. For example, a regimen can include administration periods that include 2, 3, 5, 7, 10, 15, 21, 28, or more days. These periods can be repeated. For example, a regimen can include a set number of days as previously described where the regimen is repeated 1, 2, 3, 4, 5, 6, 7, 8, 9, 10, 11, 12, 13, or more times.

In some embodiments, the compounds described herein are formulated into pharmaceutical compositions. In an embodiment, the pharmaceutical composition comprising the compounds described herein is administered to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes.

In some embodiments, the pharmaceutical composition comprises about 30 μg to about 500 μg of a compound of this disclosure, or a pharmaceutically acceptable salt or solvate thereof. In some embodiments, the pharmaceutical composition comprises about 50 μg to about 400 μg of the compound of this disclosure, or a pharmaceutically acceptable salt or solvate thereof. In some embodiments, the pharmaceutical composition comprises about 70 μg to about 300 μg of the compound of this disclosure, or a pharmaceutically acceptable salt or solvate thereof. In some embodiments, the pharmaceutical composition comprises about 70 μg to about 100 μg of the compound of this disclosure, or a pharmaceutically acceptable salt or solvate thereof. In some embodiments, the pharmaceutical composition comprises about 80 μg to about 120 μg of the compound of this disclosure, or a pharmaceutically acceptable salt or solvate thereof. In some embodiments, the pharmaceutical composition comprises about 50 μg to about 100 μg of the compound of this disclosure, or a pharmaceutically acceptable salt or solvate thereof.

In some embodiments, the pharmaceutical composition is free of additional excipients. In some embodiments, the pharmaceutical composition further comprises one or more pharmaceutically acceptable excipients.

In some embodiments, a pharmaceutical combination is provided herein comprising a pharmaceutical composition including the compound of this disclosure further comprising commercially available drugs used in the treatment of neurodegenerative diseases, psychological, cognitive, behavioral, and/or mood disorders. In some embodiments, the pharmaceutical combination achieves a synergistic effect which can permit the use of lower dosages of one or more of the components of the combination.

In one embodiment, the compounds described herein are formulated into pharmaceutical composition capable of passing through the blood brain barrier.

### Compounds

In one embodiment, compounds are provided herein. In some embodiments, the compound are of formula (I) or formula (I′), or pharmaceutically acceptable salts or solvates thereof:

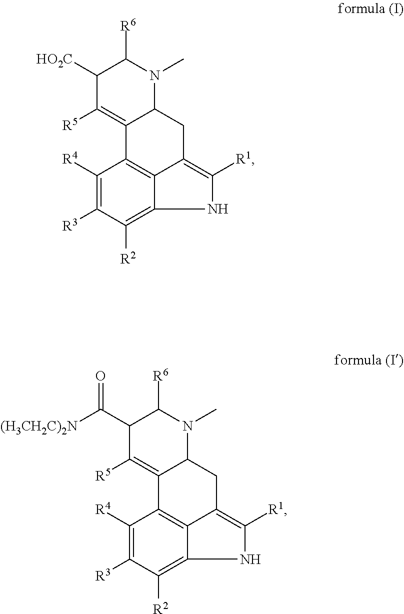

wherein R¹-R⁶ are independently selected from hydrogen, hydroxy, methoxy, a halogen, or a C₁-C₂₀ hydrocarbyl. The C₁-C₂₀ hydrocarbyl can be a C₁-C₁₅ hydrocarbyl, C₁-C₁₀ hydrocarbyl, C₁-C₅ hydrocarbyl, or C₁-C₃ hydrocarbyl. As described herein, each “hydrocarbyl” may be unsubstituted or substituted, as defined herein.

In some embodiments, at least one of R¹, R², R⁵, or R⁶ is not hydrogen. In some embodiments, at least two of R¹, R², R⁵, or R⁶ is not hydrogen. In some embodiments, at least three of R¹, R², R⁵, or R⁶ is not hydrogen. In some embodiments, each of R¹, R², R⁵, or R⁶ is not hydrogen.

In some embodiments, R³ and R⁴ are not hydrogen. In some embodiments, R³ and R⁴ are not hydrogen, and R¹, R², R⁵, or R⁶ are hydrogen.

In some embodiments, R³ and R⁴ are different. In some embodiments, R³ and R⁴ are (i) not hydrogen, and (ii) different.

In some embodiments, R³ is hydroxy. In some embodiments, R¹, R², R⁴, R⁵, and R⁶ are hydrogen, and R³ is hydroxy. In some embodiments, R³ is methoxy. In some embodiments, R¹, R², R⁴, R⁵, and R⁶ are hydrogen, and R³ is methoxy.

In some embodiments, R¹, R³, R⁴, R⁵, and R⁶ are hydrogen, and R² is methyl.

In some embodiments, R⁴ is a halo substituent (e.g., chloro-, bromo-, iodo-). In some embodiments, R¹, R², R³, R⁵, and R⁶ are hydrogen, and R⁴ is a halo substituent (e.g., chloro-, bromo-, iodo-).

In some embodiments, a compound of formula (I) and stereoisomers thereof can be used in the treatment of neurodegenerative diseases, or psychological, cognitive, behavioral, and/or mood disorders. In some embodiments, a compound of formula (I) may be further derivatized to produce an ester or amide prodrug of a compound of formula (I) or another analog of a compounds of formula (I). Such ester, amide or other analog of a compound of formula (I) may be used for the treatment of one or more neurodegenerative diseases, or psychological, cognitive, behavioral, and/or mood disorders.

### Methods

In some embodiments, methods for producing lysergic acid and derivatives thereof are provided herein.

In some embodiments, the methods include providing a compound of formula (a)—

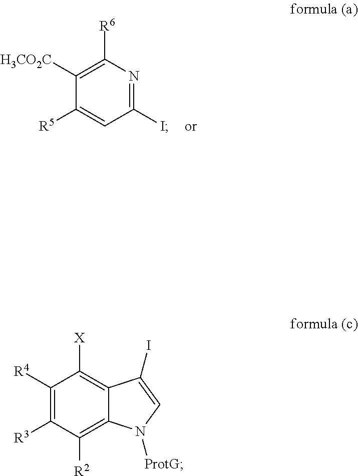

wherein R¹ to R⁶ are as defined herein. Although iodine is depicted as a substituent of formula (a), other halogens are envisioned.

In some embodiments, the methods include contacting the compound of formula (a) or formula (c) with a metal-containing compound to form a Grignard reagent. In some embodiments, the Grignard reagent is formed by contacting (i) the compound of formula (a) or formula (c) and (ii) i-PrMgCl*LiCl.

After the contacting of formula (a) or formula (c) with a metal-containing compound to form a Grignard reagent, the methods include contacting the Grignard reagent and a compound of formula (b) of formula (d), respectively, to form the compound of formula (B)—

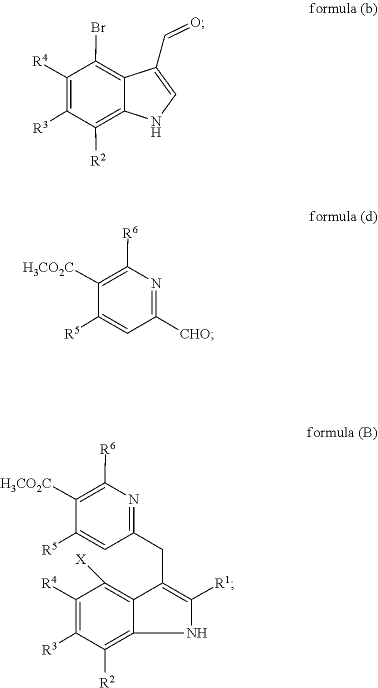

wherein R¹-R⁶ are as defined herein, and X is a halogen, such as Br.

In some embodiments, the methods include providing a compound of formula (B); contacting the compound of formula (B) with a protecting group precursor to form a protected compound comprising a protected indole nitrogen; contacting the protected compound with a methylation agent to form a methylated compound comprising a methylated pyridine nitrogen; contacting the methylated compound with a reducing agent and then a base to form the compound of formula (A). In some embodiments, the methods include contacting a compound of formula (B) with a methylation agent to form a methylated compound comprising a methylated pyridine nitrogen; contacting the methylated compound with a protecting group precursor to form a protected compound comprising a protected indole nitrogen; and contacting the protected compound with a base to form the compound of formula (A):

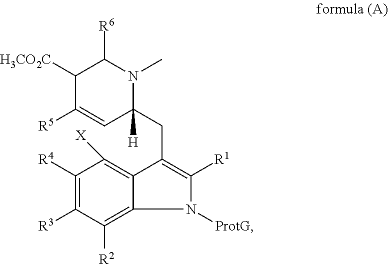

wherein R¹-R⁶ are as defined herein, ProtG is a protecting group, and X is a halogen.

Any reducing agent or base can be used in the methods described herein. In some embodiments, the reducing agent is NaBH₄, and the base is LiTMP.

In some embodiments, the methods include providing a compound of formula (A); contacting the compound of formula (A) with an annulating agent to produce an annulated compound; and contacting the annulated compound with an agent effective to remove the protecting group to form an acid of formula (I):

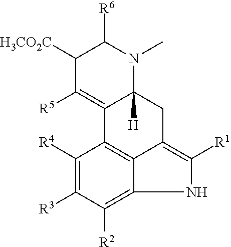

In some embodiments, the annulated compound and stereoisomers thereof can be used in the treatment of neurodegenerative diseases, psychological, cognitive, behavioral, and/or mood disorders.

In some embodiments, the annulating agent is a Heck annulating agent. In some embodiments, the annulating agent comprises tris(dibenzylidene-acetone)dipalladium(0) or bis(tri-tert-butylphosphine)palladium(0).

Any known protecting group can be used. In some embodiments, the protecting group is a tert-butyloxycarbonyl protecting group.

The agent effective to remove the protecting group can be a base, such as KOH. The protecting group can be removed by contacting the compound with aqueous KOH at a temperature of about 65° C. to about 75° C.

Any combination of the groups described above for the various variables is contemplated herein. Throughout the specification, groups and substituents thereof are chosen by one skilled in the field to provide stable moieties and compounds.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds as disclosed herein into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to the subject in need thereof.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes.

### Methods of Treatment

Described herein are methods for the treatment of neurodegenerative diseases. In some embodiments, described herein is a method of treating a neurodegenerative disease or condition including administering to a subject in need thereof an effective amount of a compound or derivatives thereof as disclosed herein.

Doses employed for adult human treatment are typically in the range of about 0.01 mg to about 5000 mg per day or from about 0.01 mg to about 1000 mg per day. In one embodiment, the desired dose is conveniently presented in a single dose or in divided doses.

### Definitions

When compounds or formulas are depicted herein with no indication of stereochemistry, the compounds or formulas are intended to read on and include all stereoisomers, and, therefore, Applicant reserves the right to claim any one or more stereoisomers of the compounds or formulas depicted or described herein.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention.

When used herein with regard to the selection of a substituent, the term “independently” indicates that (i) a substituent at a particular location can be the same or different for each molecule of a formula (e.g., (i) a compound of formula (I) can include two molecules of formula (I), with each molecule having the same or a different C₁-C₂₀ hydrocarbyl selected for R¹; and/or (ii) two differently labeled substituents selected from the same pool of substituents can be the same or different (e.g., R¹ and R² of a molecule can both be selected from “a C₁-C₂₀ hydrocarbyl”, and the C₁-C₂₀ hydrocarbyls selected for R¹ and R² can be the same or different).

The terms below, as used herein, have the following meanings, unless indicated otherwise:

“Oxo” refers to the ═O substituent. “Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. Preferably, the C₁-C₁₀ alkyl is any one of methyl, ethyl, n-propyl, isopropyl, and tert-butyl. Unless stated otherwise specifically in the specification, an alkyl group can be optionally substituted.

“Alkylene” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkoxy” refers to a radical of the formula —OR where R is an alkyl radical as defined. Unless stated otherwise specifically in the specification, an alkoxy group can be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy. The term “C₁-C₁₀ alkoxy” alone or in combination means the group C₁-C₁₀ alkyl-O—, wherein “C₁-C₁₀ alkyl” means as defined above, which includes, but not limited to, methoxy (—OCH₃), ethoxy (—OCH₂CH₃), n-propoxy (—OCH₂CH₂CH₃), iso-propoxy (—OCH(CH₃)₂), n-butoxy (—OCH₂CH₂CH₂CH₃), sec-butoxy (—OCH(CH₃)CH₂CH₃), iso-butoxy (—OCH₂CH(CH₃)₂), tert-butoxy (—OC(CH₃)₃), etc.

“Heteroalkyl” refers to an alkyl radical as described above where one or more carbon atoms of the alkyl is replaced with a O, N (i.e., NH, N-alkyl) or S atom. “Heteroalkylene” refers to a straight or branched divalent heteroalkyl chain linking the rest of the molecule to a radical group. Unless stated otherwise specifically in the specification, the heteroalkyl or heteroalkylene group can be optionally substituted. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group can be optionally substituted.

The term “aromatic” refers to a planar ring having a delocalized π-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar” (such as in “aralkyl”) is meant to include aryl radicals that are optionally substituted.

“Carboxy” refers to —CO₂H. In some embodiments, carboxy moieties can be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in some embodiments, a carboxylic acid bioisostere would ionize at physiological pH to roughly the same extent as a carboxylic acid group. Examples of bioisosteres of a carboxylic acid include, but are not limited to:

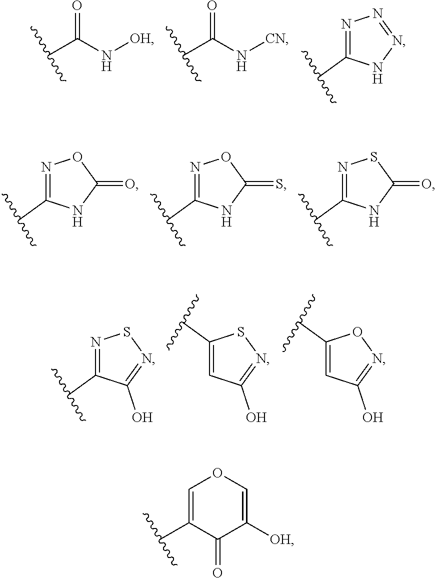

and the like.

The phrases “C₁-C₂₀ hydrocarbyl,” and the like, as used herein, generally refer to aliphatic, aryl, or arylalkyl groups containing 1 to 20 carbon atoms. Examples of aliphatic groups, in each instance, include, but are not limited to, an alkyl group, a cycloalkyl group, an alkenyl group, a cycloalkenyl group, an alkynyl group, an alkadienyl group, a cyclic group, and the like, and includes all substituted, unsubstituted, branched, and linear analogs or derivatives thereof, in each instance having 1 to about 20 carbon atoms. Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties can be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl. Examples of aryl or arylalkyl moieties include, but are not limited to, anthracenyl, azulenyl, biphenyl, fluorenyl, indan, indenyl, naphthyl, phenanthrenyl, phenyl, 1,2,3,4-tetrahydro-naphthalene, tolyl, xylyl, mesityl, benzyl, and the like, including any heteroatom substituted derivative thereof.

Unless otherwise indicated, the term “substituted,” when used to describe a chemical structure or moiety, refers to a derivative of that structure or moiety wherein (i) a multi-valent non-carbon atom (e.g., oxygen, nitrogen, sulfur, phosphorus, etc.) is bonded to one or more carbon atoms of the chemical structure or moiety (e.g., a “substituted” C₄ hydrocarbyl can include, but is not limited to, diethyl ether moiety, a methyl propionate moiety, an N,N-dimethylacetamide moiety, a butoxy moiety, etc., and a “substituted” aryl C₁₂ hydrocarbyl can include, but is not limited to, an oxydibenzene moiety, a benzophenone moiety, etc.) or (ii) one or more of its hydrogen atoms (e.g., chlorobenzene can be characterized generally as an aryl C₆ hydrocarbyl “substituted” with a chlorine atom) is substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), tertiary amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

As used herein, the terms “includes,” “is,” “containing,” “having,” and “comprises” are used in an open-ended fashion, and thus should be interpreted to mean “including, but not limited to.” When compounds or methods are claimed or described in terms of “comprising” various steps or components, the devices, systems, or methods can also “consist essentially of” or “consist of” the various steps or components, unless stated otherwise. As used herein, the term “about” means plus or minus 10% of the numerical value of the number with which it is being used.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case can be determined using techniques, such as a dose escalation study. An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The term “subject” or “patient” encompasses mammals. Examples of mammals include, but are not limited to, humans. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate. As a representative example, Applicant discloses, in some embodiments, that a compound can be contacted with KOH at a temperature of about 65° C. to about 75° C. This range should be interpreted as encompassing about 65° C. and about 75° C., and further encompasses “about” each of 66° C., 67° C., 68° C., 69° C., 70° C., 71° C., 72° C., 73° C., or 74° C., including any ranges and sub-ranges between any of these values.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### EMBODIMENTS

The following is a non-limiting listing of embodiments of the disclosure:

Embodiment 1. A method of synthesizing lysergic acid or a derivative thereof, the method comprising providing a compound of formula (A)—

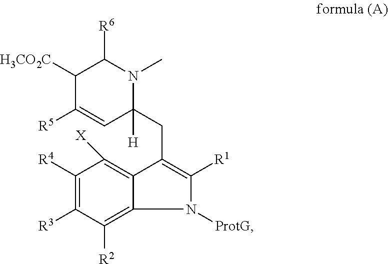

wherein ProtG is a protecting group, X is a halogen, and R¹-R⁶ are independently selected from hydrogen, hydroxy, methoxy, a halogen, or a C₁-C₂₀ hydrocarbyl, wherein the C₁-C₂₀ hydrocarbyl is unsubstituted or substituted as defined herein; and contacting the compound of formula (A) and an annulating agent to produce an annulated compound.

Embodiment 2. The method of Embodiment 1, further comprising contacting the annulated compound and an agent effective to remove the protecting group.

Embodiment 3. The method of any of the preceding embodiments, wherein the annulating agent is a Heck annulating agent.

Embodiment 4. The method of any of the preceding embodiments, wherein the annulating agent comprises tris(dibenzylidene-acetone)dipalladium(0) or bis(tri-tert-butylphosphine)palladium(0).

Embodiment 5. The method of any of the preceding embodiments, wherein the protecting group is a tert-butyloxycarbonyl protecting group.

Embodiment 6. The method of any of the preceding embodiments, further comprising providing a compound of formula (B)—

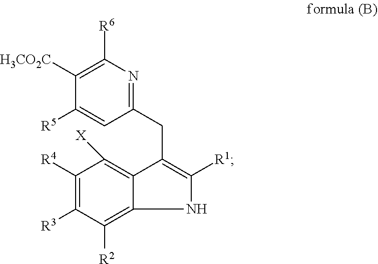

contacting the compound of formula (B), in any order, with (i) a protecting group precursor to form a protected indole nitrogen, and (ii) a methylation agent to form a methylated pyridine nitrogen; and then contacting the compound of formula (B), in any order, with (a) a reducing agent and/or (b) a base to form the compound of formula (A).

Embodiment 7. The method of any of the preceding embodiments, wherein the reducing agent is NaBH₄.

Embodiment 8. The method of any of the preceding embodiments, wherein the base is lithium tetramethylpiperidide (LiTMP).

Embodiment 9. The method of any of the preceding embodiments, further comprising providing a compound of formula (a)—

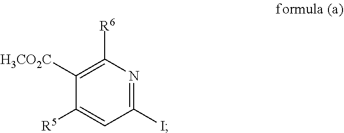

contacting the compound of formula (a) and a metal-containing compound to form a Grignard reagent; and then contacting the Grignard reagent and a compound of formula (b) to form the compound of formula (B)—

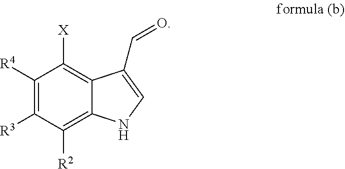

Embodiment 10. The method of any of the preceding embodiments, wherein the metal-containing compound comprises i-PrMgCl*LiCl.

Embodiment 11. The method of any of the preceding embodiments, further comprising providing a compound of formula (c)—

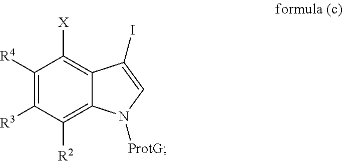

contacting the compound of formula (c) and a metal-containing compound to form a Grignard reagent; and then contacting the Grignard reagent and a compound of formula (d) to form the compound of formula (B)—

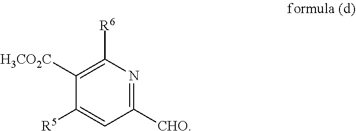

Embodiment 12. The method of any of the preceding embodiments, further comprising deacetylation to form a compound of formula (I):

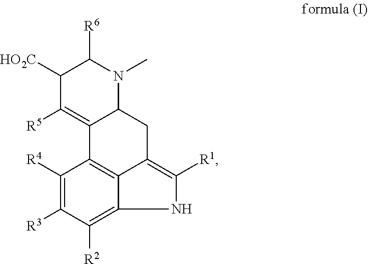

wherein R¹-R⁶ are independently selected from hydrogen, hydroxy, methoxy, a halogen, or a C₁-C₂₀ hydrocarbyl.

Embodiment 13. A compound or salt thereof formed by a method of any of the preceding embodiments.

Embodiment 14. A compound or salt thereof disclosed herein, such as any compound or salt thereof disclosed at Scheme 1, Scheme 2, Scheme 3, Scheme 4, or Scheme 5.

Embodiment 15. A compound of formula (I) or a pharmaceutically acceptable salt thereof, or formula (I′) or a pharmaceutically acceptable salt thereof:

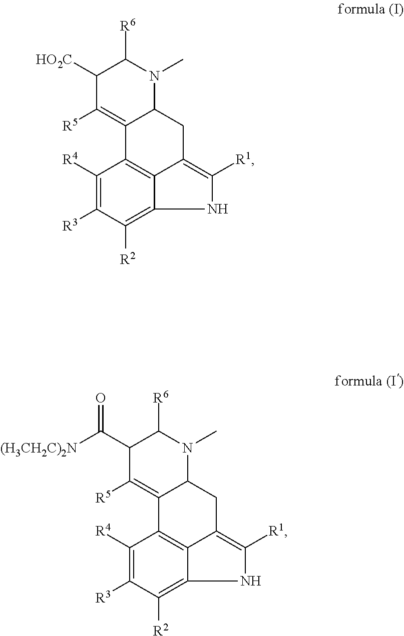

wherein R¹-R⁶ are independently selected from hydrogen, hydroxy, methoxy, a halogen, or a C₁-C₂₀ hydrocarbyl, and wherein at least one of R¹-R⁶ is not hydrogen.

Embodiment 16. The compound of any of the preceding embodiments, wherein each C₁-C₂₀ hydrocarbyl, independently, is a C₁-C₁₅ hydrocarbyl, C₁-C₁₀ hydrocarbyl, C₁-C₅ hydrocarbyl, or C₁-C₃ hydrocarbyl.

Embodiment 17. The compound of any of the preceding embodiments, wherein at least one of R¹, R², R⁵, or R⁶ is not hydrogen.

Embodiment 18. The compound of any of the preceding embodiments, wherein at least two of R¹, R², R⁵, or R⁶ are not hydrogen.

Embodiment 19. The compound of any of the preceding embodiments, wherein at least three of R¹, R², R⁵, or R⁶ are not hydrogen.

Embodiment 20. The compound of any of the preceding embodiments, wherein each of R¹, R², R⁵, or R⁶ is not hydrogen.

Embodiment 21. The compound of any of the preceding embodiments, wherein R³ and R⁴ are not hydrogen.

Embodiment 22. The compound of any of the preceding embodiments, wherein R³ and R⁴ are not hydrogen, and R¹, R², R⁵, or R⁶ are hydrogen.

Embodiment 23. The compound of any of the preceding embodiments, wherein R³ and R⁴ are different.

Embodiment 24. The compound of any of the preceding embodiments, wherein R³ and R⁴ are (i) not hydrogen, and (ii) different.

Embodiment 25. The compound of any of the preceding embodiments, wherein R³ is hydroxy.

Embodiment 26. The compound of any of the preceding embodiments, wherein R¹, R², R⁴, R⁵, and R⁶ are hydrogen, and R³ is hydroxy.

Embodiment 27. The compound of any of the preceding embodiments, wherein R³ is methoxy.

Embodiment 28. The compound of any of the preceding embodiments, wherein R¹, R², R⁴, R⁵, and R⁶ are hydrogen, and R³ is methoxy.

Embodiment 29. The compound of any of the preceding embodiments, wherein R² is methyl.

Embodiment 30. The compound of any of the preceding embodiments, wherein R¹, R³, R⁴, R⁵, and R⁶ are hydrogen, and R² is methyl.

Embodiment 31. The compound of any of the preceding embodiments, wherein R⁴ is a halogen (e.g., chloro-, bromo-, iodo-).

Embodiment 32. The compound of any of the preceding embodiments, wherein R¹, R², R³, R⁵, and R⁶ are hydrogen, and R⁴ is a halogen (e.g., chloro-, bromo-, iodo-).

Embodiment 33. The compound of any of the preceding embodiments, wherein at least one of R¹, R², R⁵, or R⁶ is not hydrogen.

Embodiment 34. The compound of any of the preceding embodiments, wherein at least one of R³ and R⁴ is not hydrogen.

Embodiment 35. The compound of any of the preceding embodiments, wherein R⁴ is C₁.

Embodiment 36. The compound of any of the preceding embodiments, wherein R⁵ is hydroxy.

Embodiment 37. The compound of any of the preceding embodiments, wherein R⁶ is methyl.

Embodiment 38. A pharmaceutical composition comprising (i) a compound or pharmaceutically acceptable salt thereof formed by a method of any of the preceding embodiments, and/or (ii) a compound or pharmaceutically acceptable salt therefore of any of the preceding embodiments.

Embodiment 39. The pharmaceutical composition of Embodiment 38, wherein the pharmaceutical composition is configured for one or more administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes.

Embodiment 40. The pharmaceutical composition of any of the preceding embodiments, further comprising one or more other components, such as one or more carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof.

Embodiment 41. A method of treating neurodegenerative disease in a patient in need thereof, comprising administering to a patient suffering from neurodegenerative disease an effective amount of a compound or a pharmaceutically acceptable salt thereof of any of the preceding embodiments, or a pharmaceutical composition of any of the preceding embodiments or any of those described herein.

Embodiment 42. The method of Embodiment 41, wherein the compound or salt thereof or pharmaceutical composition is administered in an amount of greater than about 30 μg, about 40 μg, about 50 μg, about 60 μg, about 70 μg, about 80 μg, about 90 μg, or about 100 μg.

Embodiment 43. The method of any of the preceding embodiments, wherein the compound or salt thereof or pharmaceutical composition is administered in an amount of greater than about 0.01 mg, about 0.05 mg, about 0.1 mg, about 0.5 mg, about 1 mg, about 10 mg, about 20 mg, about 60 mg, about 100 mg, about 200 mg, about 400 mg, or about 500 mg.

Embodiment 44. The method of any of the preceding embodiments, wherein the compound or salt thereof or pharmaceutical composition is administered in an effective amount from of about 10 μg to about 500 μg mg per kg of the patient's body weight (mpk).

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

### Example 1—Synthesis of Lysergic Acid

Scheme 1 depicts efforts towards implementing strategy 1 towards the synthesis of 2.

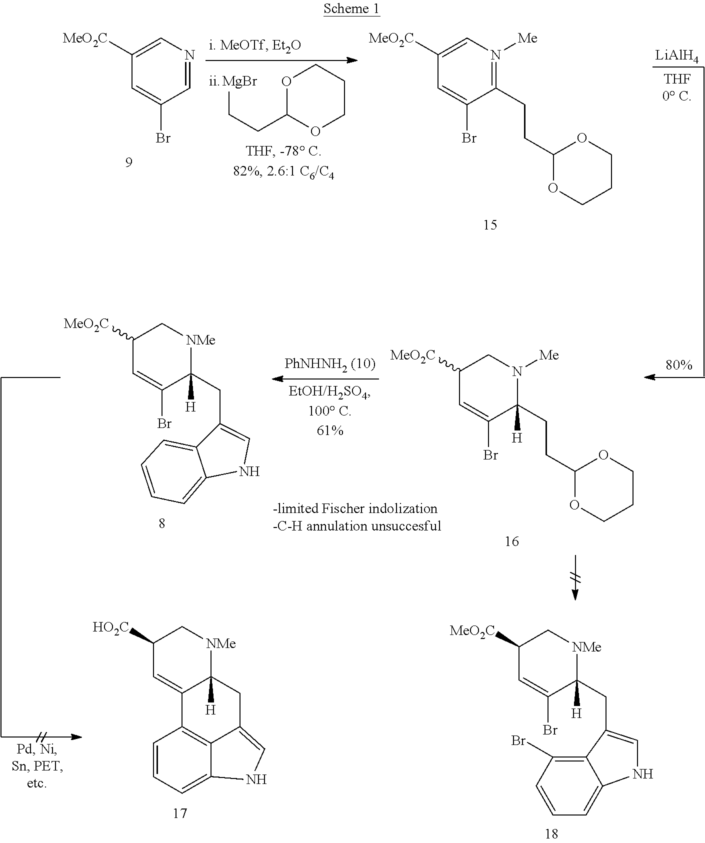

Starting from bromopyridine 9, methylation of the pyridine nitrogen with MeOTf generated an intermediate N-methylpyridinium that was subsequently trapped with Grignard reagent 11 to produce dihydropyridine 15 as the major regioisomer in a 2.6:1 C6/C4 ratio and in 82% overall yield. It was predicted that addition would be preferred at C6 due to the additive directing effect of both the ester and bromide substituents.

Next, reduction of the vinylogous carbamate in 15 with LiAlH₄ proceeded in 80% yield to deliver a 1:1 mixture of diastereomers of acetal 16. This mixture was deemed to be ultimately inconsequential for the synthesis of 2, as the alpha center to the ester is ultimately thermodynamically resolvable upon construction of the ergoline framework.

Acetal 16 was then treated with phenylhydrazine (10) and a 4% H₂SO₄/EtOH mixture at elevated temperature to afford indole 8 in 61% yield. Notably, this Fischer indolization reaction was attempted with a variety of other phenylhydrazine derivatives that gave no observable indole products. This observation impeded abilities to generate modified downstream intermediates en route to 2.

With 8 in hand, a final C—H annulation was needed to close the last six membered ring found in lysergic acid (2). Initially, following prior precedent for this kind of transformation, the only observable product was annulation at the C2 position of the indole heterocycle. Attempts to sterically deter this undesired cyclization were thwarted by an inability to properly functionalize or protect the indole nitrogen under basic conditions. This may have been due to the base sensitivity of the tetrahydropyridine ring found in 8, where the attempted conditions to deprotonate the indole nitrogen resulted in non-productive decomposition pathways. Further efforts to effect annulation of the vinyl bromide using Ni-catalysis or radical propagation (e.g. PET, Bu₃SnH and AIBN) also largely resulted in only hydrodebromination of 8.

Additionally, while a reductive coupling tactic might have been possible through an intermediate such as 18, the Fischer indolization reaction of 16 was only operable with phenylhydrazine (10), perturbing any ability to make benzenoid-functionalized indole congeners of 8. Importantly, as the same annulation disconnection would be needed to forge the key C—C bond to the C4 position of the indole, a tactical change in the placement of the halogen would likely allow for a more competent and reliable Pd-catalyzed annulation.

Scheme 2 depicts the forward implementation of the second retrosynthetic strategy towards the synthesis of 2.

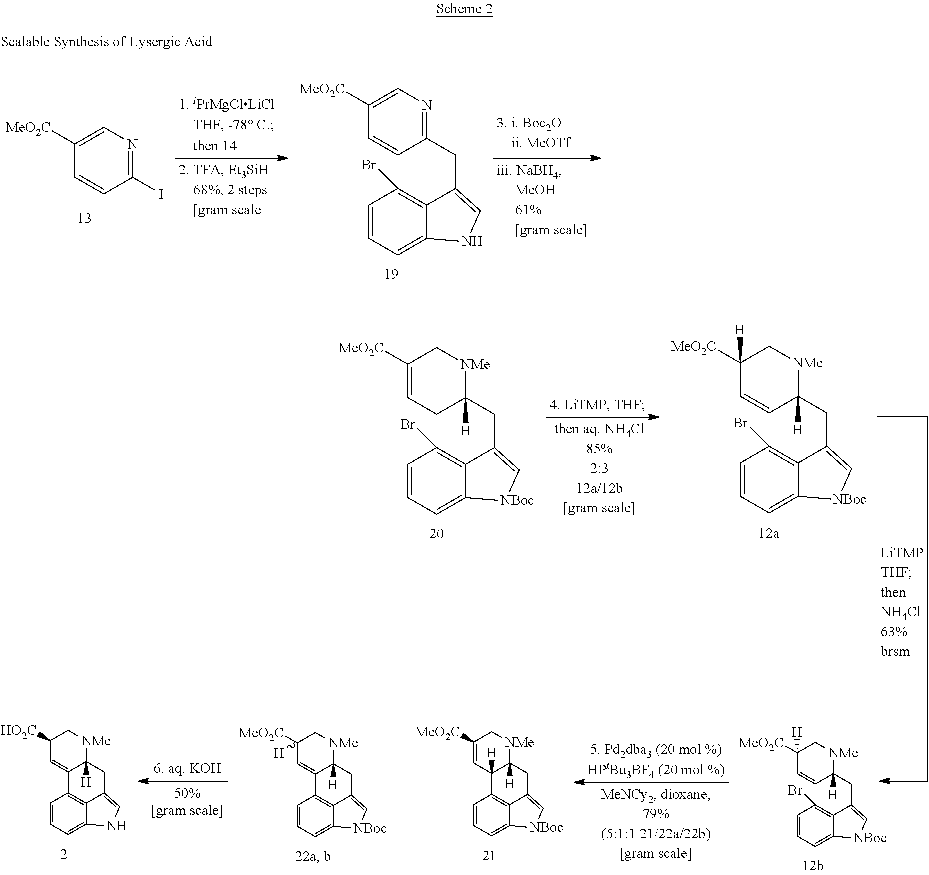

Starting from iodopyridine 13, magnesium-halogen exchange generated a heterocyclic nucleophile that was trapped with commercial aldehyde 14 to afford an intermediate alcohol (not shown) in 85% yield. Upon exposure of this intermediate to Et₃SiH and TFA, the Boc protecting group was cleaved along with subsequent reduction of the benzylic alcohol to generate indole 19 in good yield on gram scale.

The next step in the synthesis involved the reductive dearomatization of the pyridine to a tetrahydropyridine. In the event, Boc protection of 19 followed by in situ methylation of the pyridine nitrogen resulted in an intermediate pyridinium. This intermediate was not isolated, but rather treated with NaBH₄ to smoothly generate dihydropyridine 20 in 60% yield in one pot. Isomerization of the enoate with LiTMP gave a 2:3 diastereomeric mixture of 12a and 12b, of which the latter was poised for the key Heck annulation. Conveniently, 12a can be converted to 12b upon its re-exposure to the isomerization conditions with an identical diastereomeric outcome. Treatment of 12b with a catalytic amount of Fu's Pd⁰ complex (generated in situ) allowed for facile generation of 21 in excellent yield along with 22a and 22b, two diastereomeric alkene isomers, in a 5:1:1 ratio, respectively. While similar transformations have been reported, low yields and/or stoichiometric amounts of Pd have been required to effect this annulation, attesting to its challenging implementation. Furthermore, the stereochemistry of the center alpha to the ester in 12b was crucial to the success of this reaction, allowing for syn-beta-hydride elimination to proceed following migratory insertion of the putative arylpalladium(II) intermediate. Finally, saponification and isomerization of the mixture of 21, 22a, and 22b was executed as previously reported to generate lysergic acid (2) in 50% yield. See, Oppolzer, W.; Francotte, E.; Bättig, K. Total Synthesis of (±)-Lysergic Acid by an Intramolecular Imino-Diels-Alder Reaction. Preliminary Communication. Helv. Chim. Acta 1981, 64 (2), 478-481. https://doi.org/https://doi.org/10.1002/hlca.19810640212.

In conclusion, a concise synthesis of lysergic acid (2) has been accomplished in 6 steps and 14% overall yield from commercially available materials. In addition, the conversion of the final product to methyl lysergate (see U.S. Patent Application Publication NO. 2023/0116703 A1) followed by conversion to the hydrazide (see Bioorg. Med. Chem. Lett., 2008, 18, 979-982) can allow for the resolution of enantiomers (see, e.g., U.S. Pat. No. 2,447,214). Central to the brevity of this approach was the strategic, and redox-economic utilization of heteroaromatic starting materials as functionalized precursors to the ergoline core. Furthermore, while this strategic approach was initially thwarted by an insurmountable tactical conundrum, the inversion of polar synthons enabled the construction of the final tetracyclic core. This simple and potentially modular platform can permit the synthesis and investigation of efficacious psychoplastogenic LSD derivatives that will be valuable in drug discovery and psychotherapy. The molecular space that will be afforded by this synthetic blueprint holds great promise for the increased utilization of psychedelics and their derivatives as new neuropharmacological treatments.

### Example 2—Synthesis of (±)-12-Chlorolysergic Acid

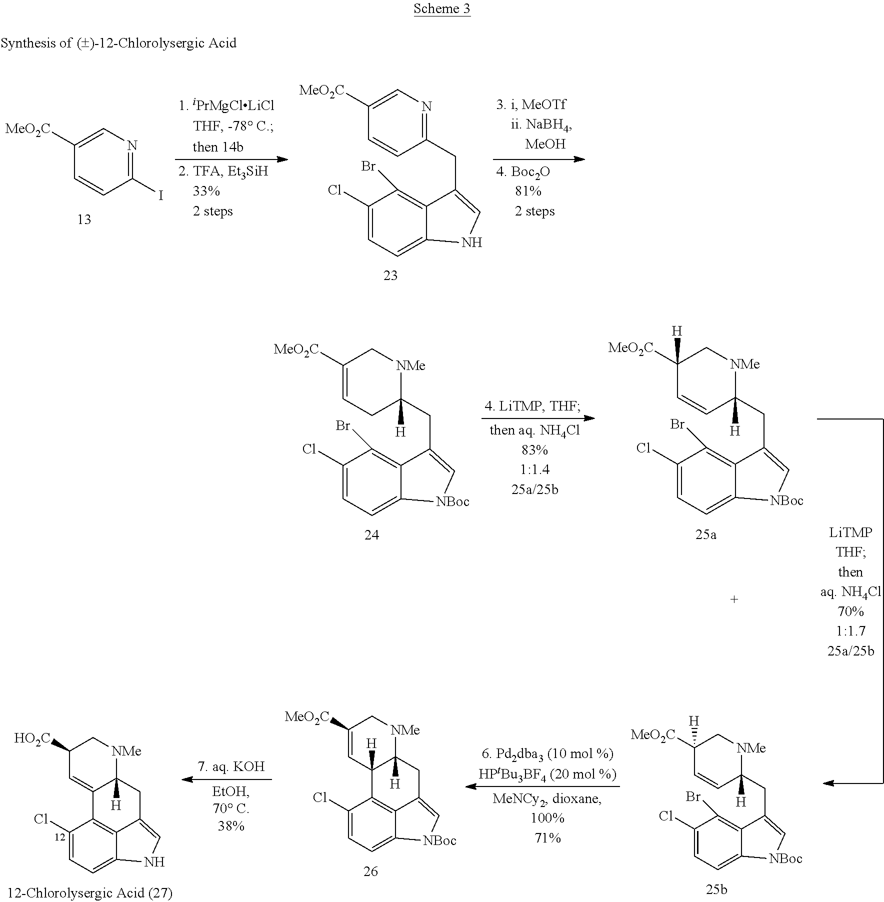

Starting from iodopyridine 13, magnesium-halogen exchange followed by addition to aldehyde 14b (i.e., 4-bromo-5-chloro-1H-indole-3-carbaldehyde) resulted in an intermediate benzylic alcohol that was reduced to give biaryl 23 in modest yield over 2 steps. Methylation and reduction of the intermediate was followed by Boc protection of the indole which proceeded in 81% yield (over 2 steps) to afford 24. This intermediate was subjected to base-mediated isomerization to give a 1:2 ratio of 25a/25b in 83% yield. The minor undesired isomer (25a) could again be recycled to afford higher quantities of 25b, which proceeded through the Heck cyclization in excellent yield to generate an enoate 26 in 57% yield. Hydrolysis of this mixture gave 12-chlorolysergic acid (27) in good yield.

### Example 3—Synthesis of (±)-13-hydroxylysergic Acid

This example discloses a route to 13-hydroxy-LSD, which is depicted below. The indolyl iodide is constructed from 3-bromo-5-methoxyaniline (B) in 3 steps. This iodide is then subjected to magnesium halogen exchange, and then quenched with commercial pyridyl aldehyde A. The resulting benzylic alcohol is reduced with TFA and triethylsilane to give the corresponding biaryl methylene. This intermediate is methylated, reduced, and Boc protected to give a corresponding dihydropyridine in 29% yield over 2 steps.

This resulting tetrahydropyridine is isomerized with LiTMP to give a prototropic isomer, which is the substrate for an intermolecular Heck cyclization to give the ergoline scaffold. Treatment of this Heck product with aq. KOH produces 13-OMe-lysergic acid, which is coupled with diethylamine and demethylated to give 13-OH-LSD. Optically pure isomers are obtained by the resolution described for that of lysergic acid above.

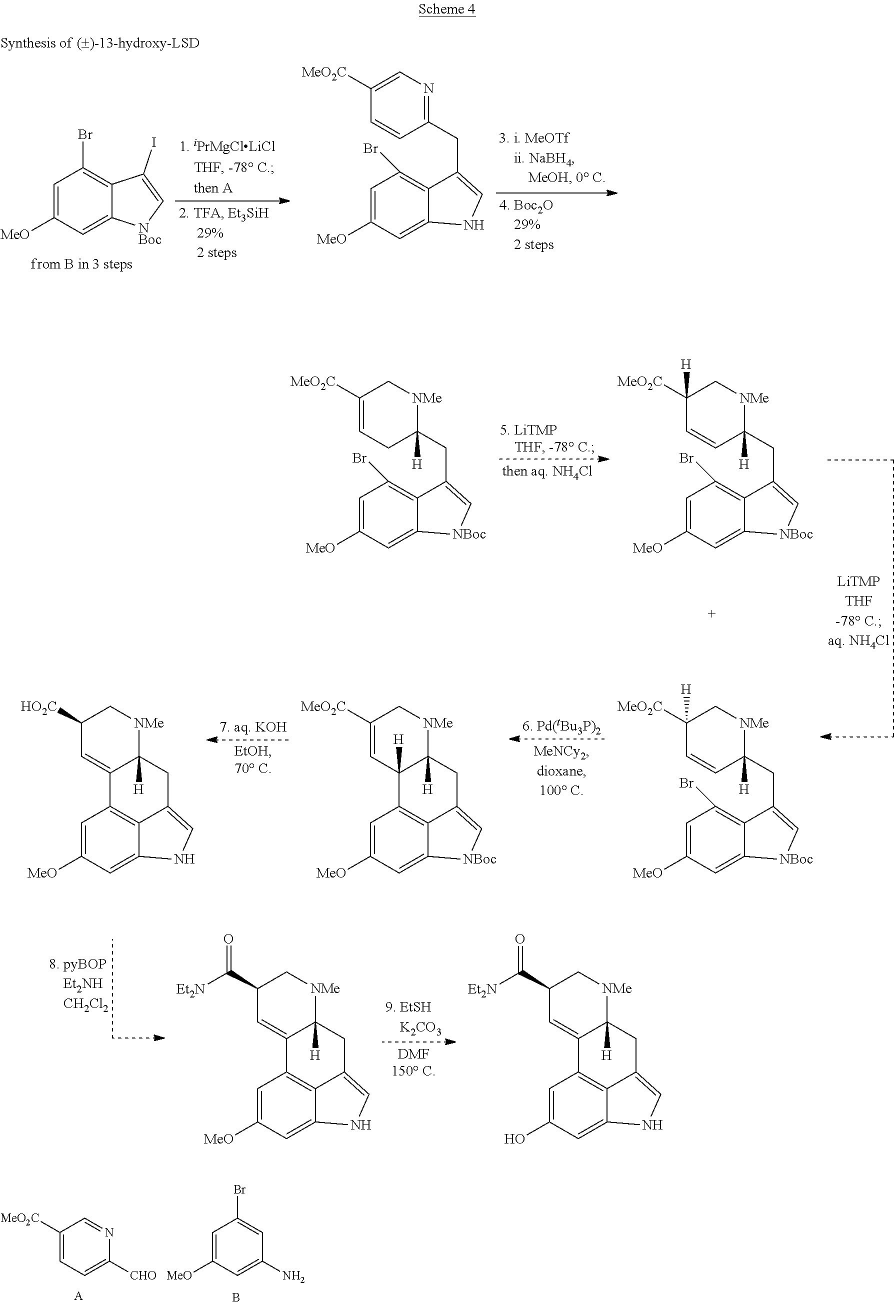

### Example 4—Synthesis of (±)-14-methyllysergic Acid

This example discloses a route to 14-Me-LSD, which is depicted below. The needed indolyl iodide is constructed from 4-bromo-2-nitrotoluene (B) in 2 steps. This iodide is subjected to magnesium halogen exchange and then quenched with commercial pyridyl aldehyde A. The resulting benzylic alcohol is reduced with TFA and triethylsilane to give the corresponding biaryl methylene. This intermediate is methylated, reduced, and Boc protected to give a corresponding dihydropyridine, which is isomerized with LiTMP to give an alkene isomer which is the substrate for an intermolecular Heck cyclization to give the ergoline scaffold. Treatment of this Heck product with aq. KOH gives 14-Me-lysergic acid, which is coupled with diethylamine to give 13-Me-LSD. Optically pure isomers are obtained by the resolution described for that of lysergic acid above.

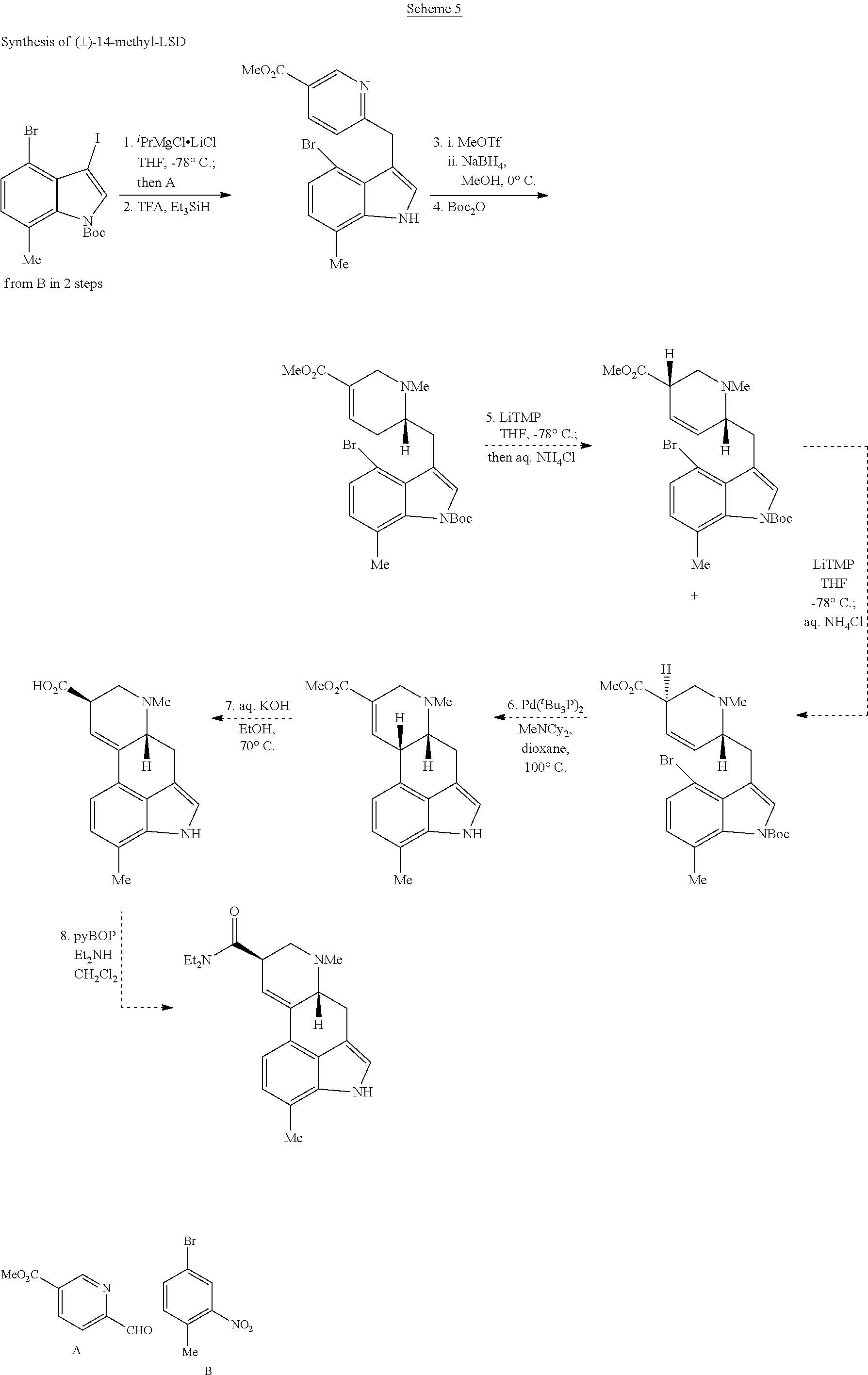

### Example 5—General Experimental/Procedural Information

All reactions were performed using flame-dried round-bottomed flasks or reaction vessels unless otherwise stated. Reactions were carried out under an inert atmosphere of nitrogen with dry solvents, unless otherwise stated. Dry benzene (PhH), dichloromethane (CH₂Cl₂), diethyl ether (Et₂O), dimethylformamide (DMF), 1,4-dioxane, methanol (MeOH), acetonitrile (MeCN), and tetrahydrofuran (THF) were obtained by passing the previously degassed solvents through activated alumina columns. Yields refer to chromatographically and spectroscopically (¹H NMR) homogeneous materials, unless otherwise stated. NMR yields were compared to an internal standard of either trimethoxybenzene or dimethyl sulfone. Reactions were monitored by thin-layer chromatography carried out on Merck silica gel plates (glass-backed, 60G, F-254) or Sigma-Aldrich aluminum oxide plates (glass-backed, F-254). Basic silica plates were prepared by treating commercial silica gel plates with 50:1 hexanes:triethylamine followed by evaporation under reduced pressure. TLC plates were visualized using ultraviolet light and an appropriate developing agent. NMR spectra were recorded on a Bruker Avance III 400, 500, or 600 MHz NMR spectrometers and were calibrated using residual solvent as an internal reference (benzene-d₆: ¹H NMR δ=7.16, ¹³C NMR δ=128.06; CDCl₃: ¹H NMR δ=7.26, ¹³C NMR δ=77.16; CD₂Cl₂: ¹H δ=5.32, ¹³C NMR δ=53.84; DMSO-d₆: ¹H δ=2.50; methanol-d₄: ¹H δ=3.31). ¹⁹F NMR spectra were calibrated using 3-fluoropyridine as an internal reference (¹⁹F NMR δ=−125.7). The following abbreviations or combinations thereof were used to explain the multiplicities: s=singlet, d=doublet, t=triplet, q=quartet, m=multiplet, br=broad, and app=apparent. When analyzing dihydropyridine products, CDCl₃ was passed through a pad of K₂CO₃ prior to its use. Crude NMR spectra were analyzed to determine the ratio of dihydropyridine regioisomers. Unisolated regioisomers were assigned based on selected peaks in the crude ¹H NMR. Flash column chromatography was performed using VWR silica gel (irregular, 60 Å, 40-60 μm), prepared basic silica gel, or deactivated aluminum oxide (alumina, for chromatography, neutral, Brockman I, 50-200 μm, 60 Å). Basic silica gel was prepared by treating commercial silica gel with 50:1 hexanes:triethylamine followed by evaporation under reduced pressure. Deactivation of alumina to Brockman grade III alumina was accomplished by adding pure water (5% w/w) to solid aluminum oxide. The mixture was shaken and then allowed to sit and equilibrate overnight in a sealed container. Preparatory TLC was performed on Merck silica gel plates (glass-backed, 60G, F-254), basic silica plates, or Sigma-Aldrich aluminum oxide plates (glass-backed, F-254). High-resolution mass spectra (HRMS) were recorded on an Agilent 6230 TOF-MS spectrometer (DART). Reagents were purchased and used without further purification, unless otherwise stated. All reagents were purchased from the suppliers below. 2,2′-Azobis(2-methylpropionitrile) was recrystallized from methanol.
Acros Organics: borane dimethylsulfide complex
Amresco: magnesium chloride (anhydrous)
Cambridge Isotope Laboratories: chloroform-d₁, acetone-d₆.
Fisher Scientific: ammonia hydroxide (conc.), phenylhydrazine, phosphorous oxychloride, pyridine, sodium chloride, sodium nitrite, and zinc metal.
Macron Fine Chemicals: sodium hydroxide.
Oakwood Chemical: allyltributylstannane, di-tert-butyldicarbonate, 4-(dimethylamino)pyridine, 4,4′-Di-tert-butyl-2,2′-bipyridyl, lithium aluminum hydride, magnesium sulfate, methyl chloroformate, methyl triflate, sodium bicarbonate, and 2,2,6,6-tetramethylpiperidine.
Sigma Aldrich: n-butyllithium (2.5M in hexanes), copper(I) iodide, N,N-dicyclohexylmethylamine, N,N′-diethylenediamine, hydrogen peroxide, iodine, isopropylmagnesium chloride lithium chloride solution (1.3M in THF), lithium chloride, pyridinium chlorochromate, sodium borohydride, sodium iodide, triethylsilane, trifluoroacetic acid, trifluoromethylsulfonic acid, tris(dibenzylideneacetone) dipalladium(0), tri-tert-butylphosphonium tetrafluoroborate.
Spectrum Chemicals: celite.
Strem: bis(cyclooctadiene)nickle(0).
Synquest Laboratories:
VWR: ammonium chloride
Ward's Science: hydrochloric acid and sulfuric acid
Methyl 6-iodonicotinate 173157-33-0 AA Blocks $140/g
Methyl 6-bromonicotinate 26218-78-0 AA Blocks $2.48/g
4-Bromo-3-formyl-1H-indole, N-BOC protected 303041-88-5 AA Blocks $91/g
4-bromoindole-3-carboxaldehyde 98600-34-1 AA Blocks $12.88/g
4-Bromoindole 52488-36-5 AA Blocks $4.67/g

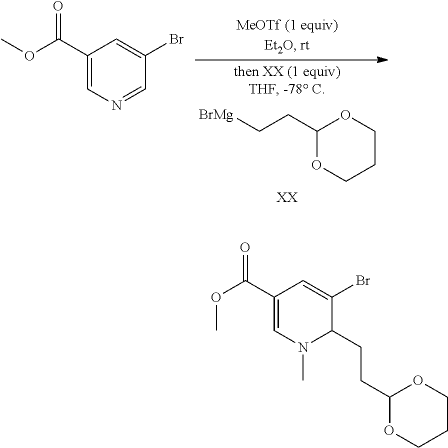

In a 30 mL test tube, pyridine XX (1.00 mmol) was suspended in Et2O (1.0 mL) and the mixture was placed under a N₂ atmosphere. The reaction vessel was placed in a rt water bath, and the mixture was stirred lightly. MeOTf (1.0 equiv) was added dropwise to the mixture and the reaction was stirred for 1 h. After the disappearance of the starting material as determined by TLC, the thick, white mixture was concentrated in vacuo and redissolved in THF (4.0 mL) before being cooled to −78° C. The Grignard reagent (1.0 equiv) was added dropwise to the reaction and the resulting yellow mixture was subjected to continuous stirring for 2 hours. The reaction was then quenched with the addition of a sat. NaHCO₃ solution (4 mL) and the mixture was allowed to warm to rt. The product was extracted with EtOAc (3×10 mL). The combined organic layer was washed with brine (15 mL), dried with MgSO₄, and concentrated under reduced pressure. The crude mixture was purified by flash column chromatography (gradient 4:1→7:3→3:2→1:1 hexanes:EtOAc, silica) to afford 203.9 mg (59% yield) of dihydropyridine XX.

¹H NMR (400 MHz, CD2Cl2) δ 7.26 (s, 1H), 6.71 (d, J=1.1 Hz, 1H), 4.54-4.47 (m, 1H), 4.38 (t, J=3.8 Hz, 1H), 4.05-4.00 (m, 2H), 3.75-3.67 (m, 2H), 3.60 (s, 3H), 2.96 (s, 3H), 2.02-1.94 (m, 1H), 1.85-1.65 (m, 3H), 1.65-1.55 (m, 1H), 1.29 (dtt, J=13.4, 2.7, 1.4 Hz, 1H).

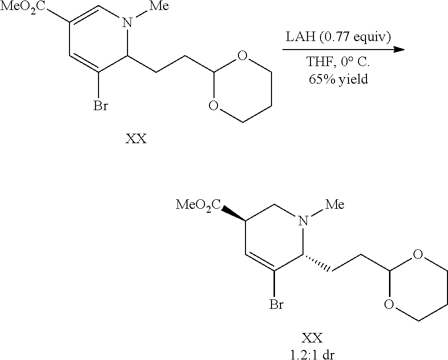

To a stirring suspension of dihydropyridine XX (1.00 g, 2.89 mmol) in THF (15 mL) at 0° C. was added a suspension of LAH (84.4 mg, 2.22 mmol) in THF (15 mL) at 0° C. over 5 min. The reaction mixture was stirred at 0° C. for 40 more min. Upon completion by TLC, the mixture was cooled to −78° C. and was then added EtOAc (7 mL) in one portion. The mixture was stirred at −78° C. for 5 min and was then warmed to 0° C. and stirred for 5 min. Then the mixture was cooled to −78° C. and then added a sat. Rochelle solution (7 mL). The mixture was stirred at −78° C. for 5 min and was then warmed to 0° C. and stirred for 5 min. To the mixture was added water (20 mL) and the product was extracted with EtOAc (3×20 mL). The combined organic layer was washed with brine (20 mL), dried with MgSO₄, and concentrated under reduced pressure. The crude mixture was purified by flash column chromatography (1:1 hexanes:EtOAc eluent, silica) to afford tetrahydropyridine XX (654 mg, 65% yield) in 1.2:1 mixture of diastereomers.

Physical State: Light yellow oil.

R_{f}=0.43 (1:1 hexanes:EtOAc eluent, silica).

¹H NMR (500 MHz, CDCl₃): δ 6.31 (q, J=1.5 Hz, 1H), 6.21 (dt, J=3.2, 1.0 Hz, 1H), 4.56-4.52 (m, 1.84H), 4.11-4.05 (m, 3.68H), 3.77-3.71 (m, 3.68H), 3.70 (s, 2.52H), 3.69 (s, 3H), 3.42-3.36 (m, 1H), 3.19-3.09 (m, 2.84), 2.96-2.91 (m, 0.84H), 2.90-2.78 (, 2.84H), 2.44 (s, 3H), 2.37 (s, 2.52H), 2.12-1.99 (m, 1.84H), 1.99-1.84 (m, 1.84H), 1.82-1.72 (m, 1.84H), 1.71-1.53 (m, 3.68H), 1.35-1.78 (m, 1.84H).

¹³C NMR (126 MHz, CDCl₃): δ 172.4, 172.1, 128.0, 126.9, 126.1, 124.6, 102.4, 102.4, 67.0, 67.0, 66.4, 66.2, 52.3, 52.3, 50.1, 46.5, 43.1, 42.5, 42.3, 39.8, 32.0, 30.2, 26.0, 25.9, 25.9, 24.7.

HRMS (DART): calc'd for C₁₄H₂₃BrNO₄⁺ [M+H]⁺ 348.0805; found 348.0804.

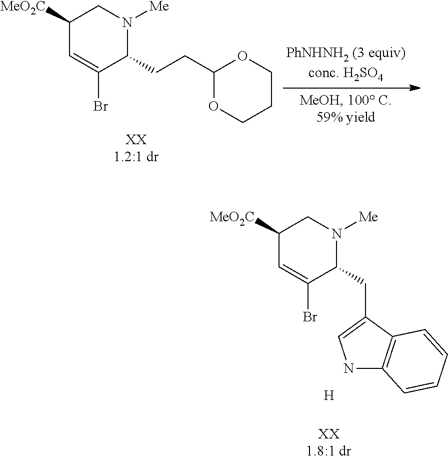

To a scintillation vial containing acetal XX (155 mg, 0.444 mmol) and phenyl hydrazine (131 μL, was added a solution mixture of 22:1 MeOH/conc. H₂SO₄ (3.0 mL). Initially, a white solid and red solution formed. The vial was flushed with N₂ and sealed with a Teflon cap. The suspension was heated and stirred at 100° C. The solid went into solution and the reaction mixture turned a deep red. The reaction mixture was heated and stirred at 100° C. for 3 h. After this time, the reaction mixture was cooled to rt and it was poured onto a 1:1 mixture of a sat. Na₂CO₃ solution and ice (6 mL). The product was extracted with EtOAc (3×20 mL). The combined organic layers were washed with brine (20 mL), dried with MgSO₄, and concentrated under reduced pressure. The crude mixture was purified by flash column chromatography (13:7 hexanes/EtOAc eluent, silica) to afford indole XX (94.7 mg, 59% yield) as a 1.8:1 mixture of diastereomers.

Physical State: Yellow oil.

R_{f}=0.39 (13:7 hexanes:EtOAc eluent, silica).

¹H NMR (600 MHz, CDCl₃): δ 8.14 (s, 0.38H), 8.10 (s, 1H), 7.70 (app. d, J=7.7 Hz, 1.38H), 7.33 (app. d, J=8.0 Hz, 1.38H), 7.18 (app. t, J=7.5 Hz, 1.38H), 7.15-7.09 (m, 2.76H), 6.39 (s, 0.38H), 6.33 (s, 1H), 3.71 (s, 1.14H), 3.69 (s, 3H), 3.54-3.44 (m, 1.38H), 3.41-3.25 (m, 4.14H), 3.22-3.11 (m, 1H), 3.09-2.8 (m, 3.14H), 2.45 (s, 4.14H).

¹³C NMR (151 MHz, CDCl₃): δ 172.2, 136.2, 136.2, 127.7, 125.8, 125.0, 122.9, 122.0, 121.9, 119.4, 119.4, 119.2, 119.1, 111.3, 111.2, 67.3, 67.3, 52.5, 52.3, 48.7, 45.9, 43.3, 42.3, 41.7, 39.5, 27.5, 26.8.

HRMS (DART): calc'd for C₁₇H₁₈BrN₂O₂⁺ [M−H]⁺ 361.0546; found 361.0515.

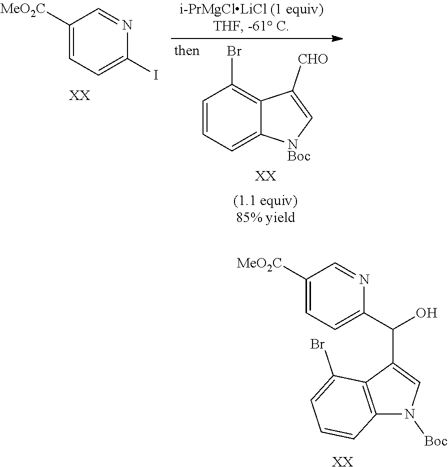

A solution of pyridyl iodide XX (1.32 g, 5.00 mmol) in THF (5.0 mL) cooled in a CHCl₃/dry ice bath (−61° C.) was charged with a solution of i-PrMgCl·LiCl (5.00 mL, 1.00 M, 5.00 mmol) over 2 min. The mixture was stirred in the cooling bath for 1 h. Upon consumption of XX as determined by TLC, the mixture was charged with a solution of XX (1.78, 5.50 mmol) in THF (3.0 mL) dropwise over 2 min. Additional THF (0.05 mL) was used to transfer any residual XX, and this solution was added dropwise over 1 min. The resulting solution was stirred in the cooling bath for 1 h. After this time, the bath was removed, and the reaction mixture was slowly warmed to rt. The reaction mixture turned a dark green upon warming and then eventually turned amber. After ˜1 h at rt. The mixture was quenched with a sat. solution of NH₄Cl (10 mL), charged with water (10 mL), and extracted with EtOAc (3×30 mL). The combined organic layers were washed with brine (15 mL), dried over MgSO₄, and concentrated under reduced pressure. The crude mixture was purified by flash column chromatography (1:1:0.05 hexanes: EtOAc: DCM eluent, silica) to afford alcohol XX (1.95 g, 85% yield)

Physical State: Light beige solid.

R_{f}=0.31 (11:9 hexanes:EtOAc eluent, silica).

¹H NMR (400 MHz, CDCl₃) δ 9.20 (s, 1H), 8.28-8.18 (m, 2H), 7.46 (s, 1H), 7.42 (d, J=7.8 Hz, 2H), 6.75 (s, 1H), 3.95 (s, 2H), 1.61 (s, 9H).

¹³C NMR (151 MHz, CDCl₃) δ 165.56, 165.17, 149.61, 148.98, 138.03, 137.17, 127.61, 127.53, 126.50, 125.53, 125.05, 122.17, 121.37, 114.72, 113.48, 84.65, 67.85, 52.52, 28.10.

HRMS (DART): calc'd for C₂₁H₂₂BrN₂O₅⁺ [M+H]⁺ 461.0707; found 461.0735.

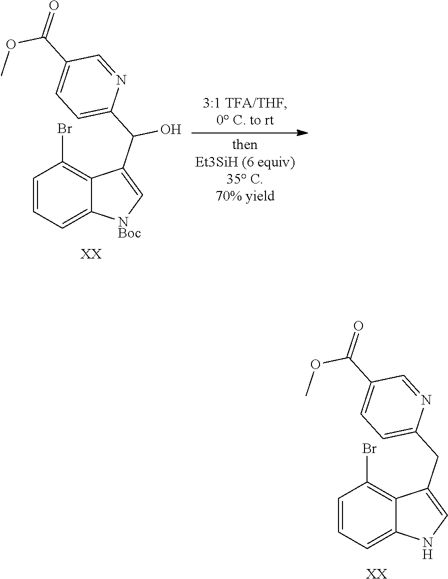

A stirring solution of carbamate XX (1.95 g, 4.23 mmol) in THF (4.2 mL) at 0° C. was charged with TFA (13 mL) in one portion turning the mixture a dark amber. The mixture was warmed to rt, and after 30 min, the mixture was charged with Et₃SiH (4.05 mL, 25.4 mmol) and stirred at 35° C. for 14 h. An additional charge of Et₃SiH (2.03 mL, 12.7 mmol) was added to the mixture, and the reaction was left to stir at 35° C. for 1 h. Upon completion as determined by 1H NMR analysis, the reaction mixture was poured into a sat. solution of NaHCO₃ (100 mL) and it was extracted with EtOAc (3×30 mL). The combined organic layers were washed with brine (15 mL), dried with MgSO₄, and concentrated under reduced pressure. The crude mixture was purified by flash column chromatography (1:1 hexanes:EtOAc eluent, silica) to afford indole XX (1.02 g, 70% yield).

Physical State: Beige solid.

R_{f}=0.35 (1:1 hexanes:EtOAc eluent, silica).

¹H NMR (400 MHz, CDCl₃) δ 9.17 (s, 1H), 8.62 (s, 1H), 8.15 (dd, J=8.2, 2.2 Hz, 1H), 7.28 (d, J=7.7 Hz, 1H), 7.23 (d, J=7.8 Hz, 1H), 7.18 (d, J=8.2 Hz, 1H), 7.04-6.96 (m, 2H), 4.65 (s, 2H), 3.93 (s, 3H).

¹³C NMR (151 MHz, CDCl₃) δ 166.74, 166.03, 150.26, 137.79, 137.60, 125.41, 125.31, 124.07, 123.54, 123.11, 122.67, 114.16, 113.12, 110.72, 52.29, 35.12.

HRMS (DART): calc'd for C₁₆H₁₄BrN₂O₂⁺ [M+H]⁺ 345.0233; found 345.0257.

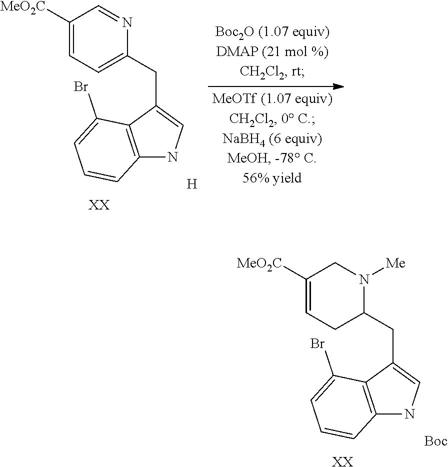

A stirring solution of indole XX (85.0 mg, 246 μmol) and Boc₂O (57.1 mg, 262 μmol) in CH₂Cl₂ (12 mL) was charged with DMAP (1.6 mg, 52.0 μmol). After 1.5 h, the reaction was complete as determined by TLC. The reaction mixture was cooled to 0° C. and was charged with MeOTf (28.7 μL, 262 μmol) in one portion. The mixture stirred for 1.5 h at 0° C. Upon completion as determined by TLC, the mixture was cooled to −78° C. A solution of NaBH₄ (56.5 mg, 1.49 mmol) in MeOH (1.2 mL) was added to the pyridinium reaction mixture over 1 min turning the reaction mixture yellow. This combined mixture stirred for 1 h at −78° C. and was then quenched with a 1:1 mixture of a sat. solution of NaHCO₃ and acetone (2 mL). The mixture was extracted with EtOAc (3×15 mL). The combined organic layers were washed with brine (15 mL), dried with MgSO₄, and concentrated under reduced pressure. The crude residue was purified by flash column chromatography (3:2 EtOAc:hexanes eluent, silica) to afford enoate XX (63.3 mg, 56% yield).

Physical State: White foam.

R_{f}=0.40 (3:2 EtOAc:hexanes eluent, silica).

¹H NMR (400 MHz, CDCl₃): δ 8.14 (d, J=8.3 Hz, 1H), 7.40 (s, 1H), 7.35 (d, J=7.7 Hz, 1H), 7.09 (t, J=8.1 Hz, 1H), 6.96 (s, 1H), 3.73 (s, 3H), 3.52-3.31 (m, 3H), 3.17 (q, J=4.6 Hz, 1H), 2.69 (dd, J=14.2, 10.0 Hz, 1H), 2.54 (s, 3H), 2.20 (s, 2H), 1.65 (s, 9H).

¹³C NMR (151 MHz, CDCl₃) δ 166.36, 149.10, 137.22, 137.03, 128.42, 128.08, 127.29, 125.80, 125.08, 117.93, 114.54, 114.07, 84.26, 56.74, 51.58, 51.03, 40.81, 28.41, 28.18, 25.87.

HRMS (DART): calc'd for C₂₂H₂₈BrN₂O₄⁺ [M+H]⁺ 463.1232; found 463.1234

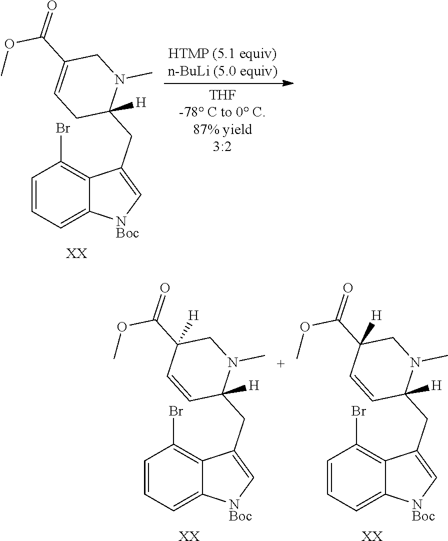

A stirring solution of HTMP (93.7 μL, 0.55 mmol) in THF (1.0 mL) was charged with n-BuLi (230 μL, 2.35 M, 0.54 mmol) at 0° C. The solution stirred for 50 min. before cooling to −78° C. after which enoate XX was added dropwise in THF (1.0 mL). The mixture changed from light yellow to amber upon addition. The reaction mixture stirred at −78° C. for 1 h and was then warmed to 0° C. and stirred for an additional 20 min. The reaction was quenched with a sat. solution of NH₄Cl (1.0 mL). This mixture was charged with water (10 mL) and extracted with EtOAc (3×15 mL). The combined organic layers were washed with brine (15 mL), dried with MgSO₄, and concentrated under reduced pressure. The crude residue was purified by flash column chromatography on neutralized silica (4:1 hexanes:EtOAc eluent, silica) to afford the minor isomer XX (16.6 mg, 33% yield) and the major isomer XX (27.2 mg, 54% yield) a 3:2 ratio.

Physical State: yellow oil

R_{f}=0.17 (1:1 hexanes:EtOAc eluent, silica).

¹H NMR (600 MHz, CDCl3) δ 7.53 (s, 1H), 7.37 (dd, J=7.8, 0.9 Hz, 1H), 7.11 (t, J=8.0 Hz, 1H), 5.89 (d, J=10.3 Hz, 1H), 5.71 (d, J=10.3 Hz, 1H), 3.70 (s, 3H), 3.59 (dd, J=14.8, 4.6 Hz, 1H), 3.48-3.42 (m, 1H), 3.30-3.25 (m, 1H), 3.20 (dd, J=11.7, 5.3 Hz, 1H), 2.86 (dd, J=15.0, 9.6 Hz, 1H), 2.68 (t, J=10.2 Hz, 1H), 2.56 (s, 3H), 1.66 (s, 9H). Iso B

1H NMR (400 MHz, CDCl₃) δ 8.17 (d, J=8.6 Hz, 1H), 7.48 (s, 1H), 7.38 (d, J=8.7 Hz, 1H), 7.11 (t, J=8.0 Hz, 1H), 5.91 (d, J=10.6 Hz, 1H), 5.75 (d, J=10.3 Hz, 1H), 3.75 (s, 3H), 3.53-3.45 (m, 1H), 3.40-3.33 (m, 1H), 3.32-3.22 (m, 2H), 2.92-2.83 (m, 2H), 2.56 (s, 3H), 1.67 (s, 9H). Iso A

¹³C NMR (151 MHz, CDCl₃) δ 172.99, 149.15, 136.92, 130.44, 128.69, 127.28, 126.27, 125.03, 123.06, 117.07, 114.50, 114.03, 84.17, 61.09, 53.99, 51.99, 43.02, 41.57, 29.27, 28.19. Iso B

¹³C NMR (151 MHz, CDCl3) δ 173.49, 149.16, 137.04, 130.42, 128.57, 127.23, 126.24, 124.98, 122.54, 117.51, 114.51, 114.02, 84.14, 60.32, 52.08, 50.31, 42.66, 38.48, 29.44, 28.20. Iso A

HRMS (DART): calc'd for C₂₂H₂₈BrN₂O₄⁺ [M+H]⁺ 463.1232; found 463.1229

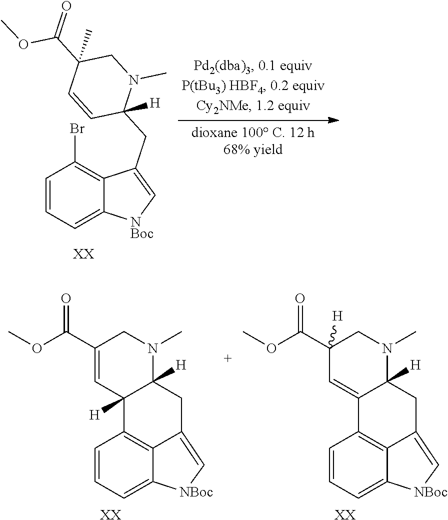

In a 9 mL reaction vial, Pd₂(dba)₃ (5.0 mg, 5.4 μmol) and P(t-Bu)₃HBF₄ (3.0 mg, 10.8 μmol) was charged with major isomer XX (25.0 mg, 0.054 mmol) in degassed 1,4-dioxane (2.7 mL) and then Cy₂NMe (13.9 μL, 64.8 μmol). The mixture was heated at 100° C. for 12 h. Upon completion as determined by TLC, the mixture was charged with water (15 mL) and extracted with EtOAc (3×15 mL). The combined organic layers were washed with brine (15 mL), dried with MgSO₄, and concentrated under reduced pressure. The crude residue was purified by flash column chromatography (1:2 hexanes:EtOAc eluent, silica) to afford an inconsequential mixture of tetracycle XX and a 1:1 diastereomeric mixture of methyl lysergate and isolysergate.

Physical State: yellow oil

R_{f}=0.23 (1:1 hexanes:EtOAc eluent, silica).

¹H NMR 1H NMR (400 MHz, CDCl3) XX δ 7.80 (s, 1H), 7.37-7.27 (m, 2H), 7.13 (d, J=7.3 Hz, 1H), 7.04 (s, 1H), 4.07 (s, 1H), 3.71 (s, 3H), 3.55-3.47 (m, 1H), 3.41-3.32 (m, 2H), 2.95 (dd, J=15.4, 4.6 Hz, 1H), 2.73-2.65 (m, 1H), 2.60 (s, 3H), 1.66 (s, 9H). Diastereomer A δ 7.80 (s, 1H), 7.37-7.27 (m, 2H), 7.13 (d, J=7.3 Hz, 1H), 6.60 (s, 1H), 3.78 (s, 3H), 3.55-3.47 (m, 2H), 3.41-3.32 (m, 2H), 2.73-2.65 (m, 2H), 2.58 (s, 3H), 1.66 (s, 9H). Diastereomer B δ 7.80 (s, 1H), 7.37-7.27 (m, 2H), 7.13 (d, J=7.3 Hz, 1H), 6.55 (d, J=4.0 Hz, 1H), 3.73 (s, 3H), 3.55-3.47 (m, 1H), 3.41-3.32 (m, 3H), 2.73-2.65 (m, 2H), 2.55 (s, 3H), 1.66 (s, 9H).

¹³C NMR (151 MHz, CDCl3) XX δ 165.97, 150.01, 140.44, 135.82, 131.51, 128.87, 126.92, 125.61, 119.99, 119.79, 115.36, 113.56, 83.41, 57.26, 51.68, 48.64, 42.22, 39.21, 36.67, 28.23.

HRMS (DART): calc'd for C₂₂H₂₇N₂O₄⁺ [M+H]⁺ 383.1970; found 383.1951

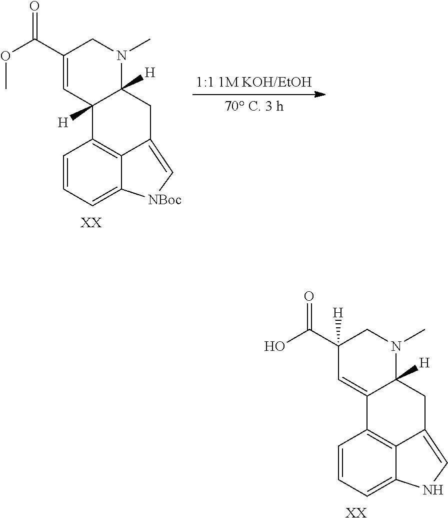

A stirring solution of compound XX (29.8 mg, 0.078 mmol) in ethanol (1 mL) and 1 N KOH (1 mL) was heated at 70° C. for 3 h. Upon completion by as determined by TLC the mixture was allowed to cool to room temperature and acidified to pH 5.8 using 1 N HCl. The resulting solution was concentrated, and the residue washed with cold water (3×1 mL) and acetone (1 mL) before being extracted with pyridine (1 mL). Evaporation of the pyridine afforded lysergic acid as a brown solid in 52% yield.

¹H NMR ¹H NMR (600 MHz, Pyr) δ 11.73 (s, 1H), 7.45 (s, 1H), 7.43 (d, J=8.7 Hz, 1H), 7.31-7.28 (m, 1H), 7.26 (s, 1H), 4.11-4.04 (m, 1H), 3.63 (dd, J=14.4, 6.0 Hz, 1H), 3.58-3.53 (m, 1H), 3.31-3.26 (m, 1H), 2.92 (q, J=12.6 Hz, 2H), 2.52 (s, 3H).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are incorporated by reference in their entirety for all purposes.

What is claimed is:

1. A method of synthesizing lysergic acid or a derivative thereof, the method comprising:
providing a compound of formula (A)— 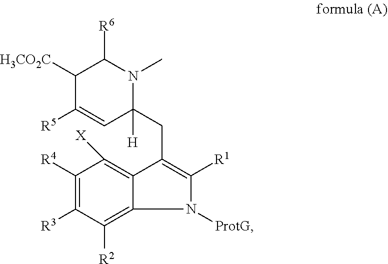
wherein ProtG is a protecting group, X is a halogen, and R¹-R⁶ are independently selected from hydrogen, hydroxy, methoxy, a halogen, or a C₁-C₂₀ hydrocarbyl, wherein the C₁-C₂₀ hydrocarbyl is unsubstituted or substituted;
contacting the compound of formula (A) and an annulating agent to produce an annulated compound; and
contacting the annulated compound and an agent effective to remove the protecting group.

2. The method of claim 1, wherein the annulating agent comprises tris(dibenzylidene-acetone)dipalladium(0) or bis(tri-tert-butylphosphine)palladium(0).

3. The method of claim 1, wherein the protecting group is a tert-butyloxycarbonyl protecting group.

4. The method of claim 1, further comprising:
providing a compound of formula (B)— 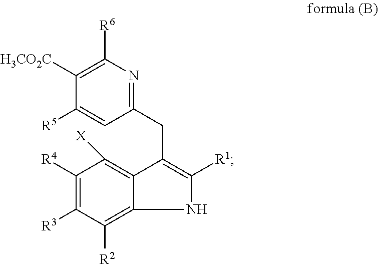
contacting the compound of formula (B), in any order, with (i) a protecting group precursor to form a protected indole nitrogen, and (ii) a methylation agent to form a methylated pyridine nitrogen; and then
contacting the compound of formula (B), in any order, with (a) a reducing agent and/or (b) a base to form the compound of formula (A).

5. The method of claim 4, wherein the reducing agent is NaBH₄, and the base is lithium tetramethylpiperidide (LiTMP).

6. The method of claim 4, further comprising:
providing a compound of formula (a)— 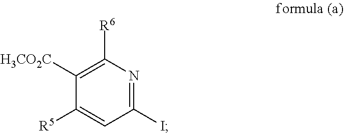
contacting the compound of formula (a) and a metal-containing compound to form a Grignard reagent; and then
contacting the Grignard reagent and a compound of formula (b) to form the compound of formula (B)—

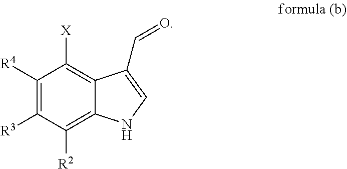

7. The method of claim 6, wherein the metal-containing compound comprises i-PrMgCl*LiCl.

8. The method of claim 4, further comprising:
providing a compound of formula (c)— 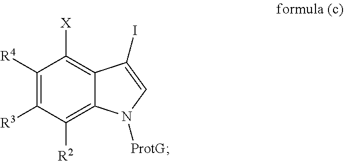
contacting the compound of formula (c) and a metal-containing compound to form a Grignard reagent; and then
contacting the Grignard reagent and a compound of formula (d) to form the compound of formula (B)—

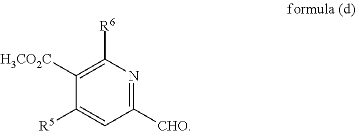

9. The method of claim 1, further comprising deacetylation to form a compound of formula (I): 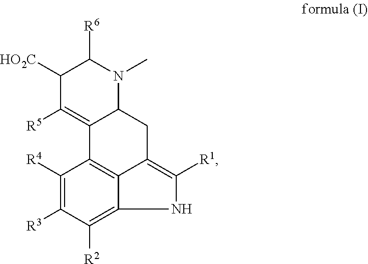
wherein R¹-R⁶ are independently selected from hydrogen, hydroxy, methoxy, a halogen, or a C₁-C₂₀ hydrocarbyl, wherein the C₁-C₂₀ hydrocarbyl is unsubstituted or substituted.

10. A compound of formula (I) or a pharmaceutically acceptable salt thereof, or formula (I′) or a pharmaceutically acceptable salt thereof: 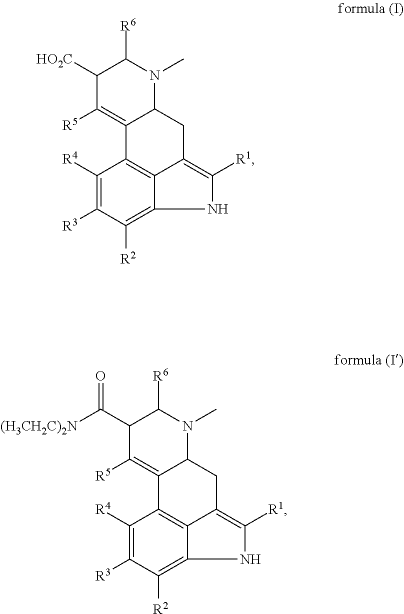
wherein R¹-R⁶ are independently selected from hydrogen, hydroxy, methoxy, a halogen, or a C₁-C₂₀ hydrocarbyl, wherein the C₁-C₂₀ hydrocarbyl is unsubstituted or substituted, and wherein at least one of R¹-R⁶ is not hydrogen.

11. The compound of claim 10, wherein at least one of R², R³ or R⁴ is a halogen, hydroxy, methoxy, or a C₁-C₆ hydrocarbyl.

12. The compound of claim 10, wherein R⁴ is Cl.

13. The compound of claim 10, wherein R⁵ is hydroxy, or R⁶ is methyl.

14. A pharmaceutical composition comprising the compound or the pharmaceutically acceptable salt of claim 10.

15. A method of treating neurodegenerative disease in a patient in need thereof, comprising administering to a patient suffering from neurodegenerative disease an effective amount of compound of formula (I) or a pharmaceutically acceptable salt thereof, or a compound of formula (I′) or a pharmaceutically acceptable salt thereof: 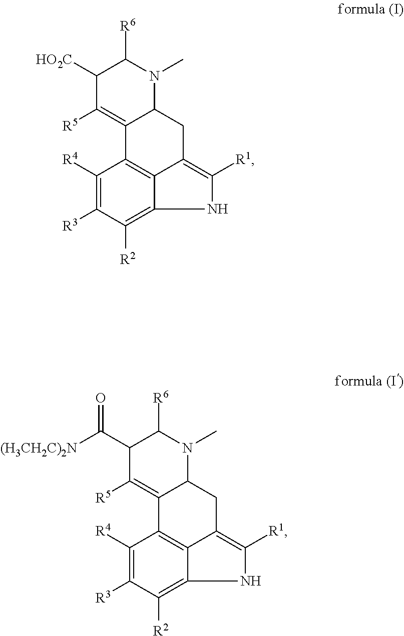
wherein R¹-R⁶ are independently selected from hydrogen, hydroxy, methoxy, a halogen, or a C₁-C₂₀ hydrocarbyl, wherein the C₁-C₂₀ hydrocarbyl is unsubstituted or substituted.

16. The method of claim 15, wherein at least one of R², R³ or R⁴ is halogen, hydroxy, methoxy, or a C₁-C₆ hydrocarbyl.

17. The method of claim 16, wherein R⁴ is Cl.

18. The method of claim 15, wherein R⁵ is hydroxy.

19. The method of claim 15, wherein R⁶ is methyl.

20. The method of claim 15, wherein (i) R¹, R², R⁴, R⁵, and R⁶ are hydrogen, and R³ is hydroxy or methoxy, or (ii) R¹, R³, R⁴, R⁵, and R⁶ are hydrogen, and R² is methyl.
